(19) United States
(12) Patent Application Publication
(10) Pub. No.: US 2011/0224491 A1
(43) Pub. Date: Sep. 15, 2011

(54) DEVICE AND METHOD FOR DEPLOYING CAP ON ENDOSCOPE

(75) Inventor: Pankaj Jay Pasricha, Cupertino, CA (US)

(73) Assignee: The Board Of Trustees Of The Leland Stanford Junior University, Palo Alto, CA (US)

(21) Appl. No.: 13/044,484

(22) Filed: Mar. 9, 2011

Related U.S. Application Data
(60) Provisional application No. 61/312,901, filed on Mar. 11, 2010.

(51) Int. Cl. A61B 1/00

(52) U.S. Cl. 600/127

(57) ABSTRACT

A system is disclosed for assisting in endoscopic procedures. The method comprises a cap and a device for deploying a cap on the endoscope while the endoscope is positioned within a body lumen. A method of deploying a cap on an endoscope during an endoscopic procedure is disclosed. The method comprises providing a cap and a device for deploying the cap on the endoscope while the endoscope is positioned with a body lumen, advancing the endoscopic within a body lumen and deploying the cap on the endoscope.

### CROSS-REFERENCE TO RELATED APPLICATIONS

This application claims priority to U.S. provisional application Ser. No. 61/312,901, filed Mar. 11, 2010, entitled Device and Method for Deploying Cap on Endoscope, which is incorporated by reference herein.

### FIELD OF THE INVENTION

The present invention relates to a system for assisting in endoscopic procedures in general, and more particularly, to a device and method for deploying a cap on an endoscope.

### BACKGROUND OF THE INVENTION

Endoscope caps are commonly used accessories during endoscopy. The endoscope cap (also referred to herein as “cap”) or hood creates a confined and readily accessible space that is useful for a variety of diagnostic and therapeutic procedures. Endoscopic caps are typically fitted onto the distal end of the endoscope. Endoscopic caps are available in a variety of shapes, forms and compositions. Straight caps, oblique caps and soft caps are examples of endoscopic caps. These endoscopic caps are most commonly used for endoscopic mucosal resection. Other uses, however, include hemostasis, foreign body removal, magnifying endoscopy, screening colonoscopy and detection axis.

While the endoscopic cap does have its uses, it is not without its disadvantages. For one thing, it is more difficult to maneuver an endoscope within a confined space when an endoscope cap is installed. For another, the endoscope cap can fill up with fluid and debris, making it difficult to visualize the desired location for a procedure. Even without such material obscuring vision, the presence of a cap can cause spatial disorientation, constraining the forward view and thus slow the advancement of the endoscope. For these reasons, endoscopic caps are used only when required. In most cases, however, an endoscopist (i.e., the doctor performing the endoscopy procedure) rarely knows in advance that an endoscope cap is needed. If such a need is encountered, the endoscopist must withdraw the endoscope, fit the cap onto the endoscope and reinsert the endoscope. These steps are inconvenient and time consuming and can potentially pose additional risk to the patient from withdrawal and reinsertion of the endoscope.

It would thus be advantageous to provide a system that will overcome the disadvantages with the conventional caps described above.

### SUMMARY OF THE INVENTION

In accordance with an embodiment of the present invention, a system is disclosed for assisting in endoscopic procedures comprising: a cap; and a device for deploying the cap on an endoscope while the endoscope is within a body lumen.

In accordance with another embodiment of the present invention, a method is disclosed for deploying a cap on an endoscope during an endoscopic procedure, the method comprising: providing a cap and a device for deploying the cap on the endoscope while the endoscope is positioned within a body lumen; advancing the endoscopic within a body lumen; and deploying the cap on the endoscope.

In accordance with an embodiment of the present invention, a cap is disclosed for deployment on an endoscopic comprising: a first ring portion configured to move along a distal end of the endoscope from a retracted position to a telescoping position extending from the distal end of the endoscope.

In accordance with another embodiment of the present invention, a device is disclosed for deploying a cap on an endoscope while the endoscope is positioned within a body lumen, the device comprising: a shaft configured to move within a lumen of the endoscope; and a portion attached to the shaft, the portion configured to contract and expand radially with respect to the shaft.

### BRIEF DESCRIPTION OF THE DRAWINGS

FIGS. 1-8 depict several stages or steps of using a system for assisting in endoscopic procedures in accordance with an embodiment of the present invention, the system including a device for deploying a cap on an endoscope.

FIGS. 9-12 depict certain cross-sectional views of the system depicted in FIGS. 1-8.

FIG. 13 depicts an enlarged cross-sectional view of the system in FIG. 11 taken along circular dotted line 13-13.

FIG. 14 depicts an enlarged cross-sectional view of the system in FIG. 11 wherein a ring portion of an endoscopic cap is shown in an extended telescoping position.

FIG. 15 depicts an enlarged cross-sectional view of the system depicted in FIGS. 1-8 in accordance with an alternative embodiment of the present invention.

FIG. 16 depicts an enlarged cross-sectional view of the system depicted in FIG. 15 wherein a ring portion of an endoscopic cap is shown in an extended telescoping position.

FIGS. 17 and 18 depict a cross-sectional view of cap on an endoscope in accordance with another embodiment of the present invention.

### DETAILED DESCRIPTION OF THE INVENTION

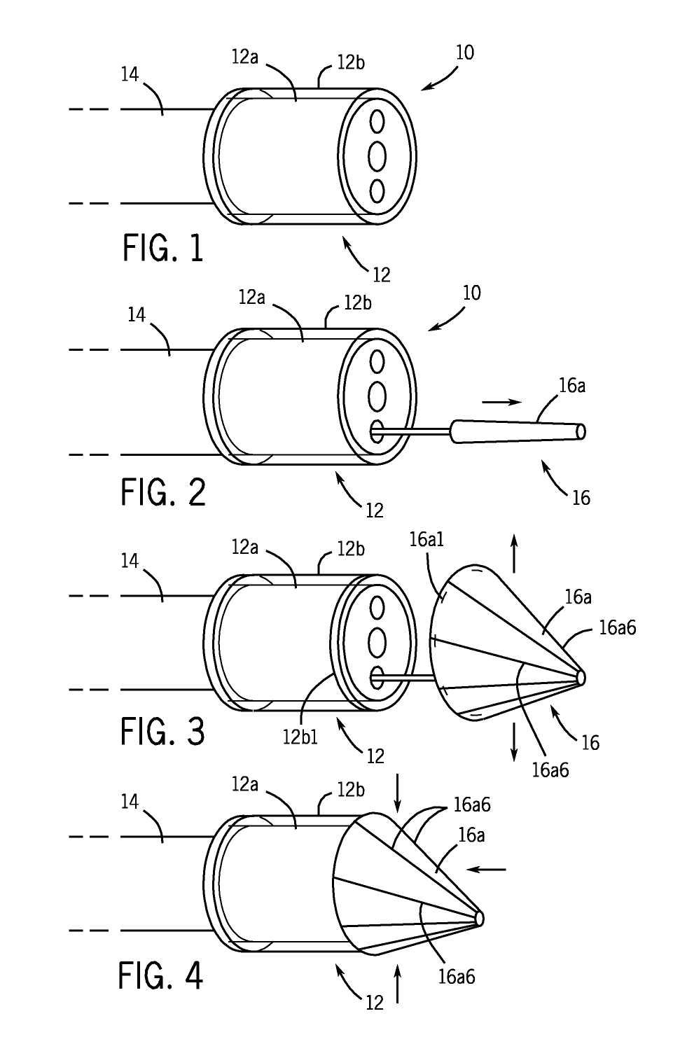

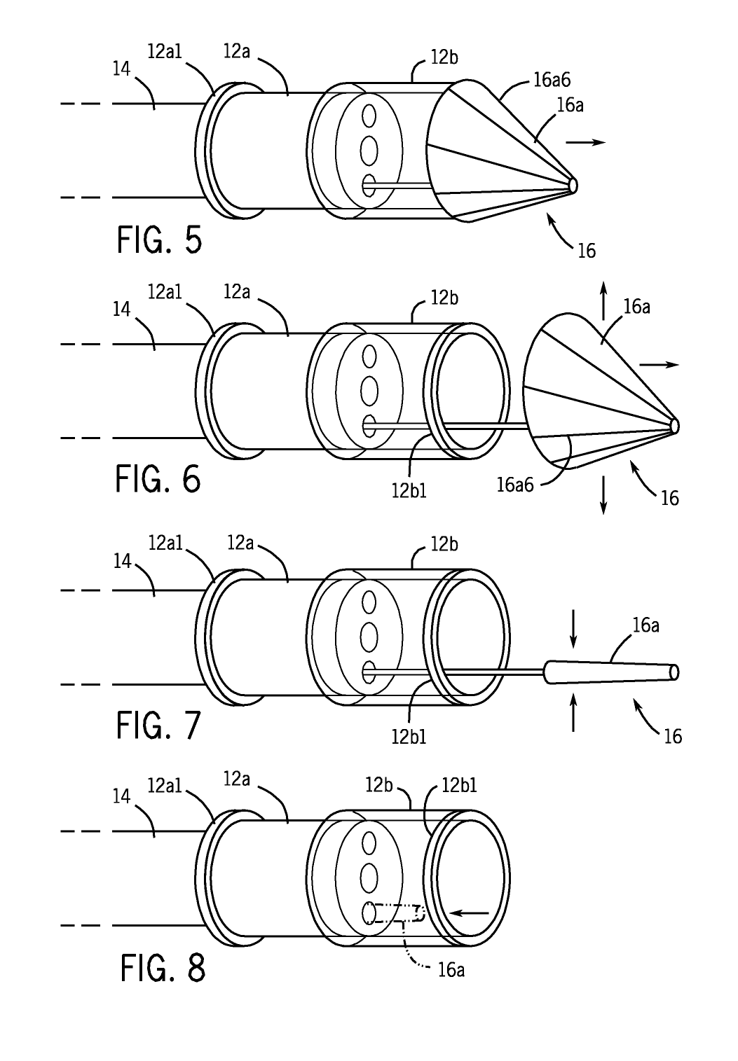

FIGS. 1-8 depict several stages or steps of using a system 10 for assisting in endoscopic procedures in accordance with an embodiment of the present invention, system 10 including a device for deploying a cap on an endoscope. System 10 includes endoscopic cap 12 mounted on endoscope 14 and device 16 for deploying cap 12 (also referred to herein as “deploying device 16”) in accordance with an embodiment of the present invention. (Deploying device 16 is not shown in FIG. 1.) Endoscope cap 12 is adapted to be advanced in a telescoping configuration. To this end, cap 12 has two ring portions 12a and 12b. Ring portion 12a is preferably mounted directly to the outer surface of the distal end of endoscope 14 by means of friction or any biocompatible adhesive that is safe to use inside a patient. It will be clear to those skilled in the art, after reading this disclosure, however, that ring portion 12a may be molded or mounted in other ways to endoscope 14. Ring portions 12a and 12b may be more easily perceived in FIGS. 5-8 wherein ring 12b is advanced in a telescoping position as described below.

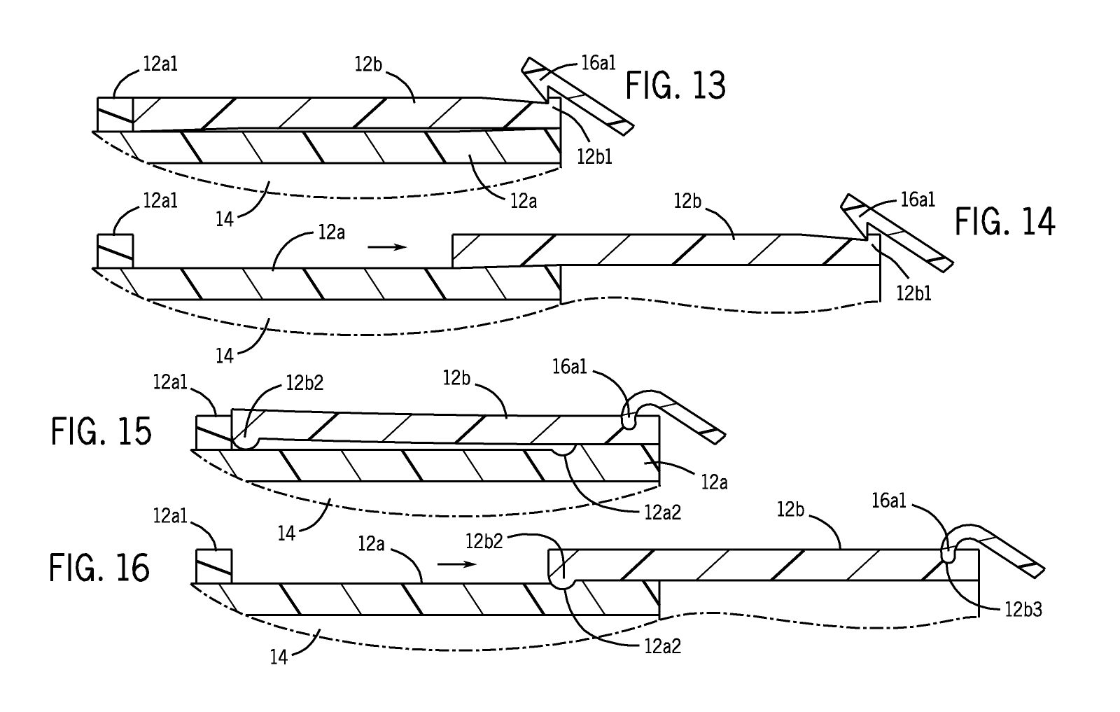

Ring portion 12a includes an annular projection or ledge 12a1 at the proximal end thereof to act as a stop for ring portion 12b. Ledge 12a1 is shown in FIGS. 1-16 but it is enlarged in FIGS. 13-16. That is, ledge 12a1 prevents ring portion 12b from sliding in the reverse direction down endoscope 14. Ledge 12a1 is shown as having the same profile as ring portion 16, but it will be clear to those skilled in the art, after reading this disclosure, that ledge 12a1 may have a different profile than ring portion 12a. That is, ledge 12a1 may project to a lesser degree (or greater) than the ring portion 12b. In addition, it will be clear to those skilled in the art, after reading this disclosure, that ledge 12a1 may extend annularly around entire ring portion 12a or alternatively may project only over a portion thereof. For example, ledge 12a1 may be constructed in the shape of bullet or any other form, so long as it acts as a stop as described above. Ring portion 12b includes a projection 12b1 that extends annularly around the distal end thereof. Projection 12b1 is designed to be engaged by deployment device 16 as discussed in more detail below. FIG. 1 depicts ring 12b of endoscopic cap 12 in a retracted position.

In the embodiment shown in FIGS. 1-14, ring portion 12a is preferably constructed to protrude minimally from endoscope 14. That is, ring portion 12a has a relatively thin profile with respect to the surface of endoscope 14. Ring portion 12a has a constant inner diameter that is slightly larger than the diameter of the endoscope 14. However, ring portion 12a has an outer diameter (from surface) that gradually increases from the proximal end to the distal end thereof (adjacent the distal end of endoscope 14). Ring portion 12b has an inner diameter that also increases from the proximal end (adjacent body section of endoscope 14) to its distal end thereof (adjacent distal end of endoscope 14). That is, the proximal end of ring portion 12b has a thickness that is larger at the proximal end than the general thickness at the distal end thereof. This is best seen in FIGS. 13 and 14. Thus, because of these diameters, the outer (surface) surface of ring portion 12a and inner surface of ring portion 12b are constructed to engage in a frictional manner when ring 12b is moved into a telescoping or expanded position as shown for example in FIG. 14. More specifically, ring portion 12b is adapted to slide on ring portion 12a along endoscope 14 in an axial direction in a telescoping configuration so that it extends beyond the distal end of endoscope 14 in an expanded position (much like the cups used for camping). Ring portion 12b is ultimately prevented from sliding off the distal end of endoscope 14 by the friction between the inner surface of ring portion 12b and outer surface of ring portion 12a. Thus, ring portion 12b is secured in place when it reaches the distal end of endoscope forming a cup with respect to endoscope 14 for endoscopic procedures. Ring portion 12b is also adapted to retract back to its original position as shown in FIG. 1 (and enlarged in FIG. 13).

While the rings 12a and 12b are sized to create friction to secure ring portion 12b in place (over ring 12a) in a telescoping position, it will be clear to those skilled in the art, after reading this disclosure, that other constructions or mechanisms are possible to secure ring portion 12b in place to create the (expanded) cup shape. For example, ring portion 12a may include an annular indentation 12a2 (i.e., recess) that extends radially near the edge thereof as shown in FIGS. 15 and 16. Annular indentation 12a2 is shown slightly offset from distal end of endoscope 14. Ring portion 12b may also include an annular projection 12b2 that extends axially along the proximal edge thereof that cooperates with (size and shape) the indentation 12a2 to cause ring portion 12b to be secured in place when it is extended along ring portion 12a in a telescoping position. As ring portion 12b slides longitudinally along ring portion 12a, annular projection 12b2 moves toward and into the annular indentation 12a2 on ring portion 12a.

Annular indentation 12a2 and cooperating annular projection 12b2 are shown as circular in shape (half circles) in FIGS. 15 and 16, but it will be clear to those skilled in the art, after reading this disclosure that any shape may be used to secure ring portion 12b in place including, without limitation a rectangular shape. In addition, the indentation and cooperating projection may not be entirely annular. That is, they may be constructed as only partially annular. That is, they may extend over only a portion of rings 12a and 12b, respectively. FIGS. 15 and 16 will be discussed again below.

It is noted that ring portion 12b is preferably clear and transparent to enable the endoscopist to see through ring portion 12b via the endoscope when ring portion 12b is in use during the endoscopic procedure, i.e., when ring portion 12b in an expanded telescoping position as described herein in a body lumen of the patient (to avoid tunnel vision). However, it will be clear to those skilled in the art that ring portion 12b may be any color or material (even translucent) to enable the endoscopist to perform the procedure.

While cup 12 includes two ring portions, it will be clear to those skilled in the art, after reading this disclosure, that cup 12 may include only one ring portion or any number of ring portions to accomplish the same results.

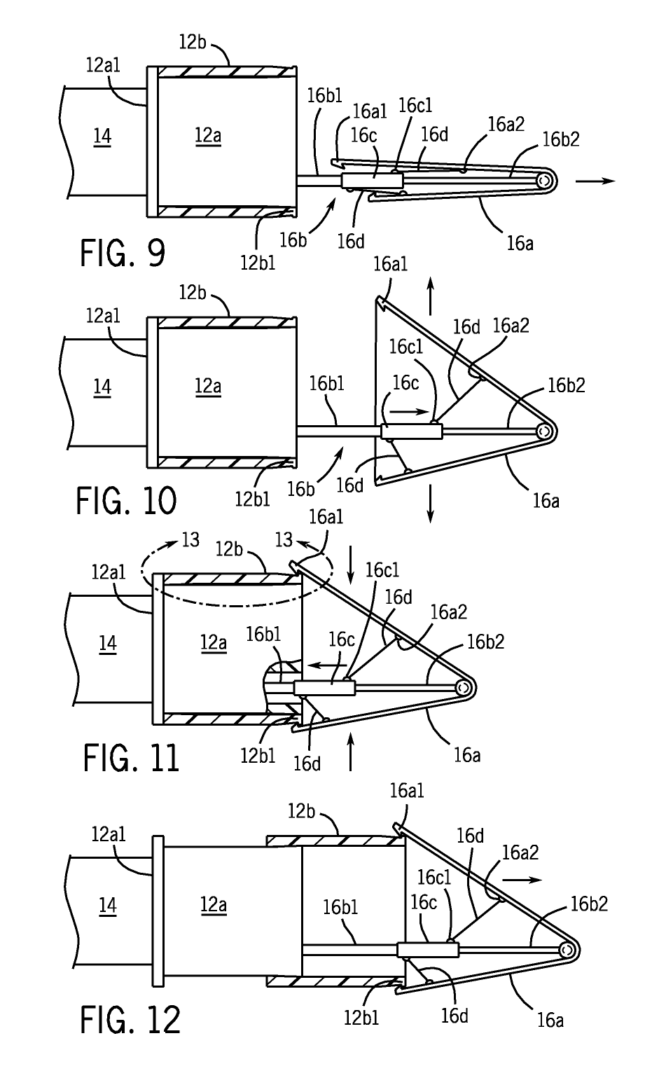

Returning to FIG. 1, ring portion 12b is shown in a retracted position. If cap 12 is required for the endoscopic procedure, an endoscopist will then advance deployment device 16 through a biopsy channel or lumen for the cap 12 deployment. This is shown in FIG. 2 and in cross-section in FIG. 9. Deployment device 16 includes umbrella portion 16a, and it is shown extending out of the biopsy channel in FIG. 2, but umbrella portion 16a is shown in a contracted position (i.e., not expanded). The construction and the operation of umbrella portion 16a are discussed below in detail with respect to FIGS. 9-12. The endoscopist then causes umbrella portion 16a to expand outwardly. Umbrella portion 16a is shown fully expanded as shown in FIG. 3 and in cross-section in FIG. 10.

The endoscopist then pulls on deployment device 16 to cause deploying device 16 to return or retract axially along and toward the distal end of endoscope 14. In the fully expanded position, proximal end (adjacent projection 16a1) of umbrella portion 16a extends over the distal end of ring portion 12b of cap 12. This is discussed more fully below.

At this time, the endoscopist manipulates deployment device 16 to cause umbrella portion 16a to contract inwardly toward ring portion 12a and endoscope 14 as shown in FIG. 4 and in cross-section in FIG. 11. As discussed more fully below, umbrella portion 16a includes projection 16a1 that extends annularly around the proximal edge of umbrella portion 16a. This is shown more clearly in FIGS. 11 and 13. Projection 16a1 is preferably shaped in the form of a shape similar to a hook and it is designed to engage and catch projection 12b1 on ring portion 12b. Projection 16a1 may alternatively be constructed in any shape to engage ring portion 12b.

Once projection 16a1 is maneuvered to slide and fit underneath annular projection 12b1 of ring portion 12b, the endoscopist then advances, i.e., pushes deployment device 16 to translate axially within the biopsy lumen of endoscope 14. This causes ring portion 12b to translate along with umbrella portion 16 in and to a telescoping position. This is shown in FIG. 5 and in cross-section in FIG. 12. At this point, ring portion 12b is expanded in a fully telescoping position. Ring portion 12b is thus secured in position by the friction between outer surface of ring portion 12a and inner surface of ring portion 12b as indicated above. As also indicated above, other alternative embodiments may be used to secure ring portion 12b in place.

At this stage, the endoscopist will manipulate the deploying device 16 so that umbrella portion 16a expands outwardly in a radial direction to release ring portion 12b. Then, the endoscopist will advance deploying device 16 axially inside the biopsy lumen of endoscope 14 so that umbrella portion 16a clears the distal end of ring portion 12b as shown in FIG. 6. The endoscopist will then manipulate deploying device 16 to cause umbrella portion 16a to contract completely as shown in FIG. 7, and will withdraw or retract deploying device 16 from the biopsy lumen as shown in FIG. 8. At this point, endoscopic cap 12 is ready for use during the medical procedure.

Once the endoscopist completes the part of the medical procedure wherein endoscopic cap 12 is needed, the endoscopist will again advance deploying device 16 down biopsy lumen (as before) to a position wherein umbrella portion 16a clears the distal end of ring portion 12b (i.e., the umbrella portion 16a extends beyond the distal end of ring portion 12b). This is similar in the steps to deployment of the cap described above. The endoscopist will manipulate deploying device 16 to expand umbrella portion 16a and pull deploying device 16 toward ring portion 12b to cause umbrella portion 16a to engage ring portion 12b and retract it. The endoscopist will maneuver umbrella portion 16a such that projection 16a1 is positioned to engage and hook projection 12b1 of ring 12b. The endoscopist then will pull the deploying device 16 toward the distal end of endoscope 14. The force against ring portion 12b will cause ring portion 12b to move to a retracted position. Alternatively, umbrella portion 16a may be used to cause ring portion 12b to retract merely by the pulling force (without the need to have projection 16a1 engage projection 12b1 of ring 12b).

Now, once ring portion 12b is fully retracted, the endoscopist will advance deploying device 16 in the opposite direction, i.e., down the biopsy lumen whereby umbrella portion 16a will move away from the end of endoscope 14. The endoscopist will manipulate deploying device 16 to cause umbrella portion 16a to contract, and will then withdraw the deploying device 16 completely from the biopsy lumen of endoscope 14. These steps are not shown in the figures.

Reference is made to FIGS. 9-12 wherein details of the construction and operation of deploying device 16 will be described in detail. FIGS. 9-12 depict certain cross-sectional views of the system depicted in FIGS. 1-8. (Note that ring portion 12a is shown in partial section.)

Deploying device 16 functions much like an umbrella (used to prevent rain from reaching one's body or used in unique cocktail drinks). As described above, deploying device 16 includes umbrella portion 16a and shaft 16b. Umbrella portion 16a acts as a canopy that is comprises a plurality of equally spaced ribs 16a6 embedded therein. Umbrella portion 16a is constructed of a material that enables umbrella portion 16a to expand and contract as desired. These materials must be suitable for use within a patient's body. Examples of the material include combinations of plastics such as polyvinyl chloride (PVC) and polyethylene and nitinol or other metals.

Shaft 16b includes shaft member 16b1, rod 16b2 and hub ring 16c. In this embodiment, note that the shaft 16b is positioned off-center with respect to the center of umbrella portion 16a. That is, shaft 16b is positioned such that the radius around shaft 16b to the proximal end of umbrella portion 16a (adjacent projection 16a1) is not constant. Because the endoscopic lumen is typically constructed (positioned) off-center, shaft 16b is similarly constructed off-center (with respect to umbrella portion 16a) to properly position projection 16a1 in place to engage and pull annular projection 12b1 of ring portion 12b when umbrella section 16b is contracted around ring portion 12b as described above. The endoscopist must manipulate and maneuver shaft 16b to properly position umbrella portion 16a in place to engage ring portion 12b as described herein. Those skilled in the art know however, after reading this disclosure, that shaft 12b may be positioned with respect to umbrella portion 16a as required by an endoscope design to achieve proper positioning over ring portion 12b.

As indicated above, shaft 16b also includes hub ring 16c that is preferably connected to or alternatively integrated with shaft member 16b1. Shaft member 16b1 and hub ring 16c are constructed with a channel to receive rod 16b2. Shaft member 16b1 and hub ring 16c are adapted to slide longitudinally along rod 16b2.

Deploying device 16 also includes two ribs 16d that couple hub ring 16c to umbrella portion 16a. Ribs 16d are preferably constructed as single sections or spokes that are adapted to pivot about pins 16c1 along and part of hub ring 16c and pins 16a2 along the inner surface and part of umbrella portion 16a to enable it to expand and collapse (contract) in response to translational movement of shaft member 16b1 over rod 16b2. In this embodiment, two ribs 16d are used. However, it will be clear to those skilled in the art, after reading this disclosure, that any number of ribs may be employed to achieve the desired results.

In operation, the endoscopist may advance or retract deploying device 16 within the lumen of endoscope 14 by holding and moving (pushing or pulling) both shaft member 16b1 and rod 16b2 of shaft 16b. In order to expand or collapse umbrella portion 16a, the endoscopist will hold rod 16b2 stationary and will translate (move) shaft member 16b1 over and along rod 16b2. As a result, hub ring 16c moves along with shaft member 16b1. When the endoscopist desires to expand umbrella portion 16a, he/she will move shaft member 16b2 forward toward umbrella portion 16a and away from endoscope 14. Ribs 16d will pivot about pins 16a2 of umbrella portion 16a and pins 16c1 of hub ring 16c, respectively and umbrella portion 16a will expand outwardly. When the endoscopist desires to contract or collapse umbrella portion 16a, he/she will move shaft member 16b1 away from umbrella portion 16a while holding rod 16b2 in place. Ribs 16d will then pivot about pins 16a2 and pins 16c1 and cause umbrella portion 16a to contract (collapse) inwardly.

As described above, FIG. 13 depicts an enlarged cross-sectional view of the system 10 in FIG. 11 taken along circular dotted line 13-13, and FIG. 14 depicts an enlarged cross-sectional view of the system 10 in FIG. 12 wherein ring portion 12b of an endoscopic cap 12 is shown in an extended telescoping position. The construction of ring portions 12a and 12b and the frictional engagement are shown in detail.

FIGS. 15 and 16 are briefly discussed above. FIGS. 15 and 16 depict an enlarged cross-sectional view of the system depicted in FIGS. 1-8 in accordance with an alternative embodiment of the present invention. Specifically, FIG. 15 depicts an enlarged cross-sectional view of the system wherein the ring portion of endoscopic cap is shown in a retracted position along distal end of the endoscope. FIG. 16 depicts an enlarged cross-sectional view of the system depicted in FIG. 15 wherein a ring portion of an endoscopic cap is shown in an extended telescoping position. As discussed above, projection 12b2 of ring portion 12b and indentation 12a2 cooperate in construction to secure ring portion 12b in place in a telescoping position.

Also shown in FIGS. 15 and 16 is an alternative embodiment of the mechanism for moving ring portion 12b from a retracted position to a telescoping position and back. As described above, umbrella portion 16a includes projection 16a1, but in this embodiment, it is now shaped as a round annular post (much like a candy cane) and ring portion 12b includes recess 12b3 that cooperates in shape and size of projection 16a1.

While ring portion 12b as described above employs a projection 12b1 (FIGS. 1-14) or a recess 12b3 (FIGS. 15 and 16) and umbrella portion 16a employs different two shapes for projection 16a1 to move ring portion 12b from a retracted position to a fully telescoping position and back, it will be clear to those skilled in the art, after reading this disclosure, that other alternative embodiments may be used to effect this ring portion 12b movement.

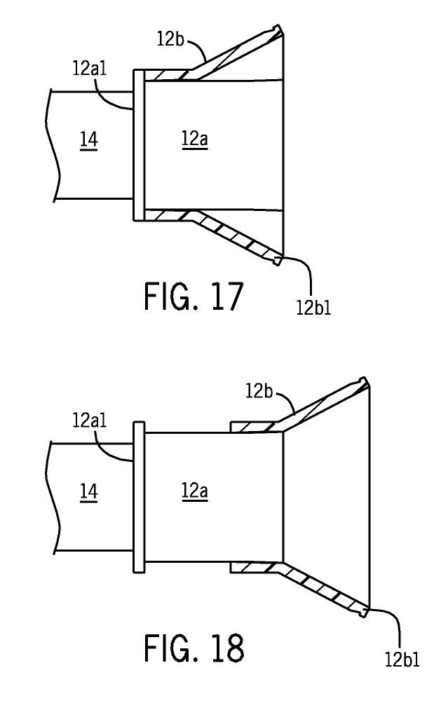

While the shape of the extendable cap 12 is shown in FIGS. 1-16 as cylindrical, it will be clear to those skilled in the art, after reading this disclosure, that cap 12 may be constructed in any configuration as required for a specific task. For example, cap 12 may be shaped in the form of a funnel as depicted in FIGS. 17 and 18. FIGS. 17 and 18 depict a cross-sectional view of cap on an endoscope in accordance with another embodiment of the present invention. (Note that ring portion 12a is shown in partial section.) As seen in these FIGS. 17 and 18, ring portion 12b includes a cylindrically shaped section and a funnel shaped section. The cylindrically shaped section is constructed to enable the cap 12 to properly slide along ring portion 12a. Ring portion 12b is shown in a retracted position in FIG. 17 and an expanded telescoping position in FIG. 18.

FIGS. 1-18 and the description depict certain embodiments of deploying device 16. However, it will be clear to those skilled in the art, after reading this disclosure, that other mechanisms may be used to deploy endoscopic cap 12 including, for example, a miniature motor or electronic mechanism.

The endoscopic cap described above has many uses as described herein, including, for example, treating gastrointestinal hemorrhage such as from varices or ulcers by suctioning the bleeding segment into the extended cap and either ligating, banding or injecting it to produce hemostasis.

It is to be understood that the disclosure teaches examples of the illustrative embodiments and that many variations of the invention can easily be devised by those skilled in the art after reading this disclosure and that the scope of the present invention is to be determined by the claims below.

What is claimed is:

1. A system for assisting in endoscopic procedures comprising:
a cap; and
a device for deploying the cap on the endoscope while the endoscope is positioned within a body lumen.

2. The system of claim 1 wherein the device is configured to translate axially with respect to the endoscope.

3. The system of claim 1 wherein the device comprises a portion configured to expand outwardly and to contract inwardly.

4. The system of claim 3 where the portion is an umbrella portion.

5. The system of claim 3 wherein the cap includes a first ring portion that is adapted to move from a retracted position along a distal end of the endoscope to a telescoping position extending from the distal end of the endoscope.

6. The system of claim 5 wherein the portion of the device includes a projection for engaging the cap.

7. The system of claim 6 wherein the projection is shaped as a hook.

8. The system of claim 6 wherein the projection is shaped as a post.

9. The system of claim 6 wherein the first ring portion includes a projection for engaging the projection of the portion of the device.

10. The system of claim 6 wherein the first ring portion includes a recess for receiving the projection of the portion of the device.

11. The system of claim 5 wherein the cap further includes a second ring portion for mounting to the distal end of the endoscope, the first ring portion configured to slide along the second ring portion in an axial direction from the retracted position to the telescoping position.

12. The system of claim 11 wherein the first and second ring portions are configured to engage in a frictional manner, to enable the first ring portion to be secured into the second telescoping position.

13. The system of claim 5 wherein the first ring portion includes a cylindrically shaped section.

14. The system of claim 13 wherein the first ring portion includes a funnel shaped section.

15. The system of claim 1 wherein the cap includes a first ring portion that is adapted to move from a retracted position along a distal end of the endoscope to a telescoping position extending from the distal end of the endoscope.

16. A method of deploying a cap on an endoscope during an endoscopic procedure, the method comprising:
providing a cap and a device for deploying the cap on the endoscope while the endoscope is positioned within a body lumen;
advancing the endoscopic within a body lumen; and
deploying the cap on the endoscope.

17. The method of claim 16 wherein deploying the cap includes advancing the device within a lumen of the endoscope to move the cap from a retracted position along a distal end of the endoscope to a telescoping position extending from the distal end of the endoscope.

18. The method of claim 17 wherein deploying the cap includes retracting the device to move the cap from the telescoping position to the retracted position.

19. The method of claim 16 wherein the device includes an umbrella portion, and wherein deploying the cap includes contracting the umbrella portion for deploying the cap inwardly to engage the cap and expanding the umbrella portion outwardly to disengage the cap.

20. The method of claim 17 wherein the cap includes a ring portion that is configured to move from the retracted position to the telescoping position.

21. The method of claim 16 wherein deploying the cap includes withdrawing the device from a lumen of the endoscope.

22. A cap for deployment on an endoscopic comprising:
a first ring portion configured to move along a distal end of the endoscope from a retracted position to a telescoping position extending from the distal end of the endoscope.

23. The cap of claim 22 wherein the first ring portion includes an annular projection along a distal end of the first ring portion.

24. The cap of claim 22 wherein the first ring portion includes a recess at a distal end of the first ring portion.

25. The cap of claim 24 further comprising a second ring portion for mounting to the distal end of the endoscope, the first ring portion configured to slide along the second ring portion in an axial direction from the retracted position to the telescoping position.

26. The cap of claim 25 wherein the first and second ring portions are configured to engage in a frictional manner, to enable the first ring portion to be secured into the telescoping position by such frictional engagement.

27. The cap of claim 22 wherein the first ring portion includes a cylindrically shaped section.

28. The cap of claim 27 wherein the first ring portion includes a funnel shaped section.

29. The cap of claim 25 wherein the second ring portion includes a ledge at the proximal end thereof to act as a stop for the first ring portion.

30. A device for deploying a cap on an endoscope while the endoscope is positioned within a body lumen, the device comprising:
a shaft configured to move within a lumen of the endoscope; and
a portion attached to the shaft, the portion configured to contract and expand radially with respect to the shaft.

31. The device of claim 30 wherein the shaft includes:
a rod;
a shaft member;
a hub ring connected to the shaft member; and
at least one rib connected between the hub ring and portion, the shaft member and hub ring configured to slide along the rod thereby causing the portion to expand and contract.

32. The device of claim 30 wherein the portion is an umbrella portion.

33. The device of claim 30 wherein the portion includes a projection for engaging the cap.
